(19) United States
(12) Patent Application Publication
(10) Pub. No.: US 2018/0328353 A1
(43) Pub. Date: Nov. 15, 2018

(54) DEVICE FOR SUCTIONING GASEOUS OR LIQUID MEDIA

(71) Applicant: EBERHARD TIMM GMBH, Buxtehude (DE)

(72) Inventor: Eberhard Timm, Buxtehude (DE)

(21) Appl. No.: 15/574,059

(22) PCT Filed: May 14, 2015
(86) PCT No.: PCT/DE2015/000235

§ 371 Date: Nov. 14, 2017

(51) Int. Cl. F04B 45/02; F04B 49/22; F04B 53/20; F04B 19/06; A61M 1/00; A61F 13/84; B08B 5/04; B08B 15/00

(52) U.S. Cl. CPC F04B 45/02; F04B 49/225; F04B 53/20; F04B 19/06; A61M 1/0035; F04B 9/14; A61M 1/0056; A61M 1/0072; B08B 5/04; B08B 15/00; A61F 13/84

(57) ABSTRACT

The invention relates to a device for suctioning gaseous or liquid media, which arise preferably in medical care, for example in medical aids, and are suctioned out of said medical aids noiselessly by means of a vertically extendable bellows (4), the bellows upper end (5) of which is fastened to an upright (1, 3) and the bellows lower end (6) of which is connected to a slidably guided loading part (2), which produces a vacuum in the interior (14) of the bellows (4) as the loading part drops under gravitational force, which vacuum forces a suctioning of the medium from the aid into the bellows (4), wherein the bellows (4) unfolds.

### PRIORITY CLAIM

This patent application is the U.S National stage under U.S.C. 371 of PCT/DE2015/000235 filed May 14, 2015 and designating the United States.

### FIELD OF INVENTION

The invention relates to a device for suctioning gaseous or liquid media which occur preferably in medical care, for example, in medical aids, and for suctioning said media noiselessly.

From DE-U-7011944, a manually activated pump for suctioning gaseous media from a completely closed pouch is known. For suctioning the gaseous media, a rubber bellows is used, which is compressed by hand and which, after release, springs back to its full bellows shape due to its own force. In the process, a low pressure is generated in the interior thereof, which is suitable for suctioning the medium from the pouch.

Such suctioning processes always occur only immediately after or for a brief duration after the compression. Regularity or uniform suctioning is not possible. This is desirable, for example, in a decubitus patients, where moist gases develop in the medical aids, which have to be suctioned. It would be possible to carry out this suctioning by means of a pump which is operated with electrical current. However, such motors are expensive and above all they generate undesirable noise. Folding bellows suction pumps have been known for a long time, for example, from DE 3822001 C1, DE 4416599 A1 and from the Utility Patent DE 1908290 U. However, they do not disclose a gravity-operated folding bellows suction pump. The aim of the invention is to enable a soundless uniform suctioning of gas over a longer time period. The aim defined is achieved according to the invention by the subject matter of Claim 1. Advantageous designs are the subject matter of the dependent claims or described below.

By means of the suctioning device according to the invention, gaseous or liquid media can be suctioned completely soundlessly. The device is particularly suitable for home care, since it is easy to operate. In the process it uses a vertically extendable bellows, the bellows upper end of which is fastened on a stand, and the bellows lower end of which is connected to a slidably guided loading part which, as it descends due to the force of gravity in the internal space of the folding bellows, generates a negative pressure which forces a suctioning of medium from the aid into the bellows, wherein the bellows unfolds.

Because the device uses the force of gravity for suctioning, the operation thereof does not require additional energies supplied from outside, the use of which generally causes noise.

According to a design of the invention, it is provided that the stand comprises three or four vertical columns, an upper device shell stationarily fastened to the columns for the attachment of the bellows upper end, and a lower device shell, the position of which can be changed, for the attachment of the bellows lower end, wherein the lower device shell descends under the load of weight due to the force of gravity and for, this purpose, is preferably slidably guided on the columns. Naturally, it is also possible to attach an additional weight to the lower device shell, which loads the lower device shell downward.

According to a design of the invention, it is provided that the bellows upper end is put from below on a collar of the upper device shell by means of an upper sliding collar and that the bellows lower end is put from above on a collar of the lower device shell by means of a lower sliding collar.

According to an additional design of the invention, it is provided that the bellows is implemented as a folding bellows and comprises a larger number of folding segments between the upper and lower sliding collars thereof, wherein in each case a ring made of a flexible planar material is connected on the outer edge thereof of larger diameter to a next adjacent ring on the outer edge of larger diameter thereof, and the other next adjacent ring is connected on the inner edge thereof with smaller diameter to the inner edge with smaller diameter of the first ring.

Preferably, the areas of the material connections of the folding bellows, in the area of the inner edge and/or of the outer edge of the rings, are provided with a stiffening, in particular a stiffening adjoining the outer edges with the larger diameter. The stiffenings which, in particular, are annular, hold the shape of the folding bellows, when the flexible planar material of the rings has low stability.

Moreover, it is provided that the upper and/or lower device shell comprise(s) an inlet and/or outlet opening. An inlet and/or outlet opening is preferably closable, for example, by means of a hinged cover and, independently thereof, it is preferably provided (at least) in the upper device shell. In this manner, the device can be brought to another emptying site, when the hinged cover is closed, without any unpleasant odor occurring. The inlet and/or outlet opening is/are closed with the hinged cover. Here, the hinged cover closes off against a sealing ring that is used.

According to an embodiment, one device shell comprises a closable inlet opening, and the lower device shell comprises a closable outlet opening. However, it is also possible to provide an opening in only one device shell and to use it as inlet and outlet opening, depending on the operating state or pressure conditions in the interior of the bellows.

According to another design of the invention, it is provided that a valve is provided in the inlet opening (which, according to a design, can optionally also be used as outlet opening), in order to set or regulate the suction volume speed of the medium to be suctioned through the inlet opening and in order to set the descent speed of the lower device shell. A pipe or tube can be connected to the inlet opening.

According to another design of the invention, it is provided that a collection bag for suctioned medium can be connected to the outlet opening (which, according to a design, can optionally also be used as inlet opening). This can be achieved, for example, by a plug-on crown which can be fixed to the outlet opening for the transfer of suctioned medium and which preferably opens the outlet opening as a result of the process of insertion.

According to another design of the invention, it is provided that the outlet opening (which, according to a design, can optionally also be used as inlet opening) also comprises a valve via which the outflow speed can be regulated, or the inlet valve at the same time is the outlet valve and, for this purpose, is set into an operating mode which allows a higher suctioning volume speed.

According to another design of the invention, it is provided that a filter and/or drying element is inserted into the inlet opening (which, according to a design, can optionally also be used as outlet opening).

The filter is replaceable and acts as absorber and/or disinfecting agent. The absorber such as activator charcoal, for example, binds odors, and the disinfecting agent neutralizes germs such as bacteria, viruses or fungi. Into the filter, upstream or downstream thereof or integrated therein, a drying agent can be used, such as, for example, calcium chloride, superabsorbent polymers, molecular mesh, bentonites, silica gel or zeolites. The drying agent can be provided with an indicator in order to indicate when it needs to be replaced. Filter and drying agent can be used as individually or jointly replaceable pads or cartridges.

According to another design of the invention, handles are provided on the lower device shell, by means of which the lower device shell can be pulled up for emptying. According to another operating mode, the device is turned upside down for this purpose, so that the movable lower device shell is at the top and descends slowly or at a moderate speed.

The invention is explained in further detail in reference to the drawings.

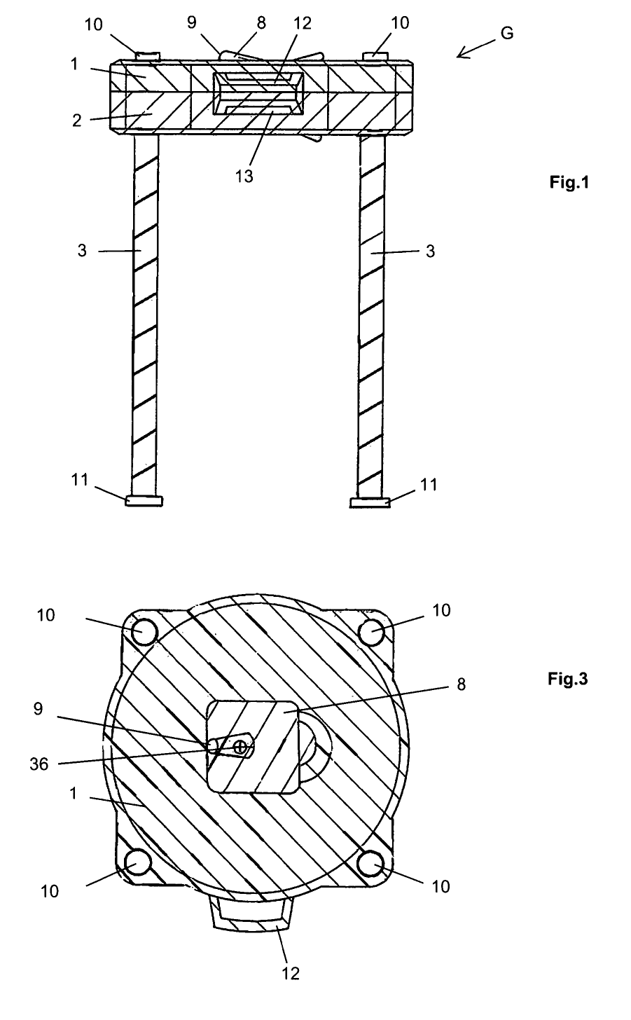

FIG. 1 shows a device for suctioning gaseous or liquid media, with an upper device shell and a lower device shell, which are arranged on four supporting columns, wherein the upper device shell is firmly connected with the supporting columns to a stand, while the lower device shell is slidable along the supporting columns, and wherein, between the device shells, an extendable folding bellows, shown in the extended state in FIG. 2, is arranged in the folded state,

FIG. 2 shows a view of the device according to FIG. 1 rotated by 45°, wherein the lower device shell has been moved downward and, in the process, it has extended the folding bellows,

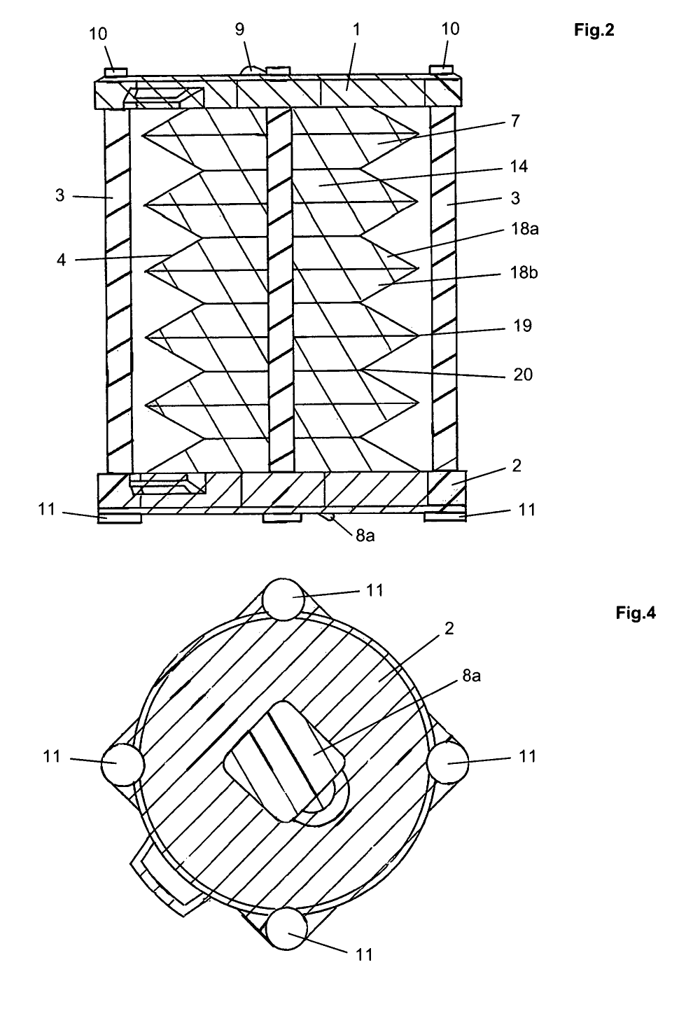

FIG. 3 shows a top view onto the upper device shell with a hinged cover which closes an inlet and outlet opening and comprises a hose connection,

FIG. 4, viewed from below, shows a top view onto the lower device shell with a hinged cover without hose connection,

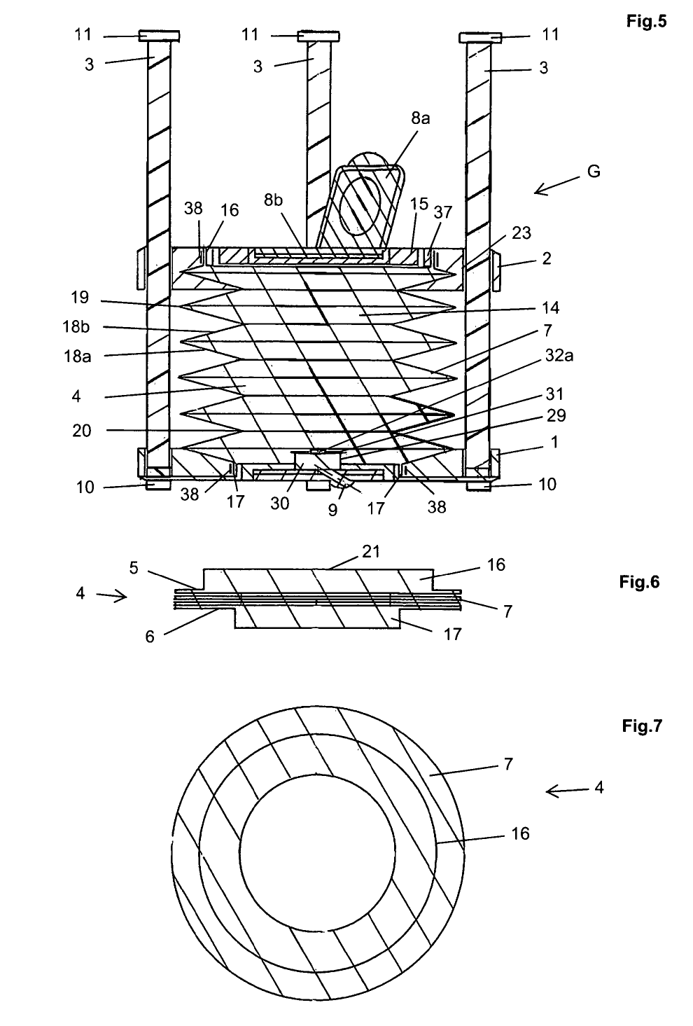

FIG. 5 shows a device according to FIG. 1 rotated by 45° with respect to the view according to FIG. 1 and turned upside down in the emptying phase, wherein the hinged cover of the lower device shell is open, and the folding bag is half folded,

FIG. 6 shows the folding bellows as folded unit with upper and lower sliding collar,

FIG. 7 shows the folding bellows according to FIG. 6 in a top view,

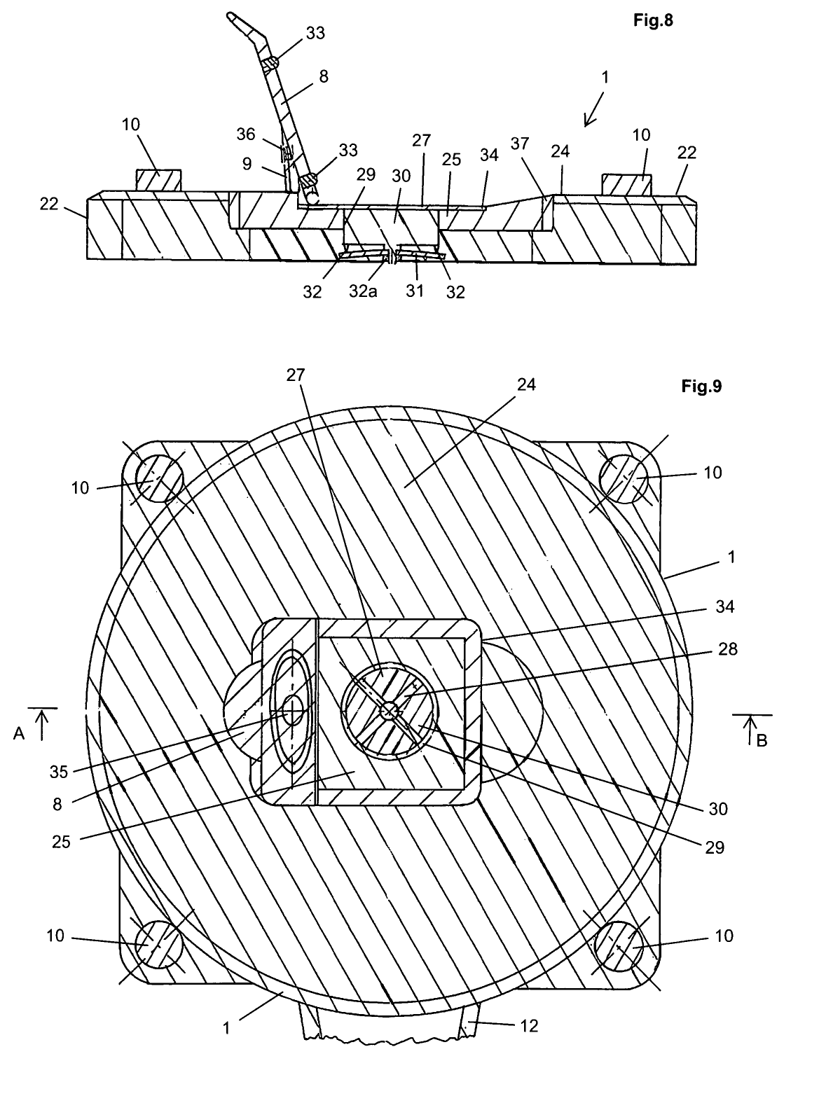

FIG. 8, in an enlarged representation and in section A-B according to FIG. 9, shows the upper device shell with hinged cover open and with a filter sleeve and a plate valve,

FIG. 9 shows a top view onto the upper device shell with open hinged cover,

FIG. 10 shows the device with partially extended folding bellows and open hinged cover, wherein handles are provided on the lower device shell, by means of which the lower device shell, which has been moved downward, can be pulled up for emptying,

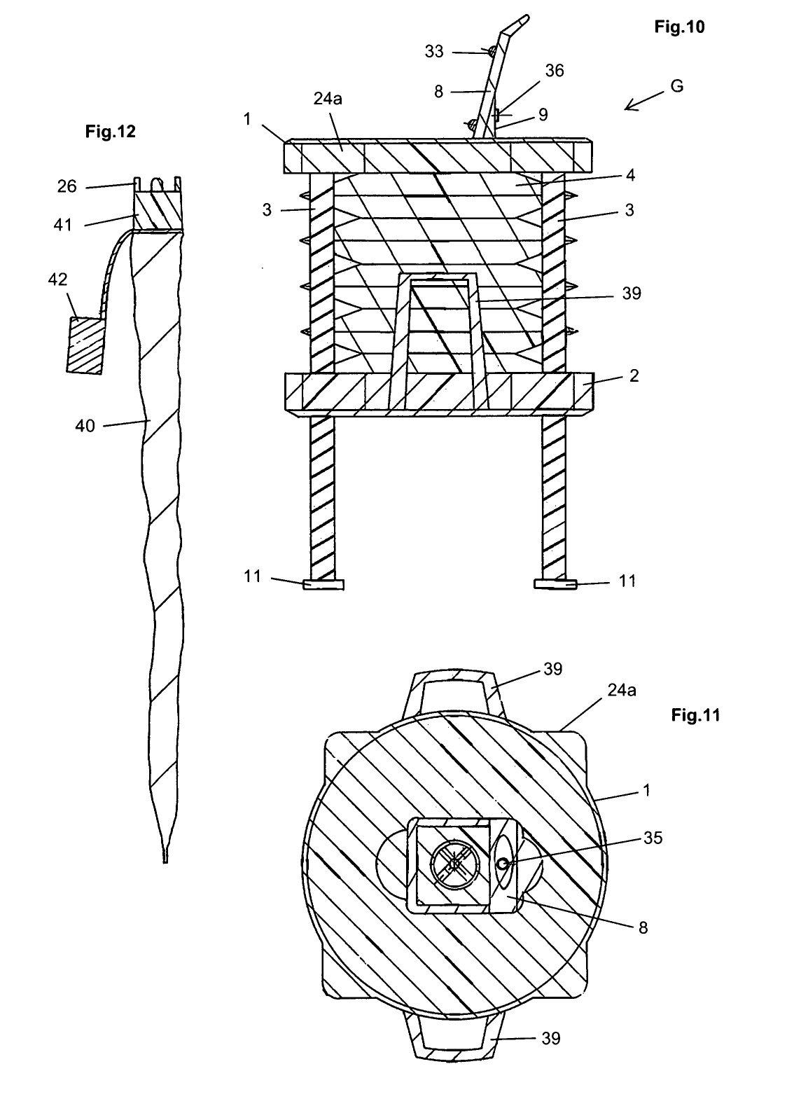

FIG. 11 shows a top view onto the device according to FIG. 10 with pull-up handles on both sides, and

FIG. 12 shows a film bag into which the medium suctioned into the folding bellows can be transferred.

In FIG. 1, a suctioning device (G) for suctioning or pumping gaseous or liquid media is represented, which can be used in medical care. Such medical care consists, for example, of the removal of moisture-laden gases from diapers. Due to the suctioning of the gas, the tendency of sores to develop due to decubitus position is decreased. In addition, by means of the suctioning device (G), it is also possible to suction bodily fluids such as saliva, and wound fluid, for example, in the case of moisture producing wounds. Here it is essential that the suctioning occurs continuously and that no motor-generated noise occurs during the suctioning.

The suctioning device (G) represented in FIG. 1 consists of an upper device shell (1), a lower device shell (2), and four vertical supporting columns (3). Three supporting columns (3), in a so-called three-leg design, would also be sufficient. The four supporting columns (3) are firmly connected with the upper device shell (1) to a stand. The lower device shell (2) is slidably guided on the supporting columns (3). Between the device shells (1) and (2), a folding bellows (4) is located, not visible in FIG. 1, but visible in different states of extension and representations in FIGS. 2, 5, 6 and 10.

As can be seen clearly in FIG. 6, the folding bag (4) has a bellows upper end (5) and a bellows lower end (6), between which holding segments (7) are located. As can be seen in FIG. 2, the bellows upper end (5) is fastened to the upper device shell (1), and the bellows lower end (6) is fastened to the lower device shell (2). The fastening of the bellows ends (5, 6) can be seen clearly from FIG. 5. In FIG. 5, the suctioning device (G) is represented, turned upside down for the purpose of emptying.

On the upper side of the upper device shell (1), a hinged cover (8) is provided with a hose connection (9), by means of which a gaseous medium is to be suctioned with a suction hose. On the upper ends of the supporting columns (3), FIG. 1 shows small rubber stoppers (10) and, on the lower ends of the supporting columns (3), large rubber stoppers (11) which are used as feet. The device shells (1 and 2) are provided with laterally protruding handles (12 and 13).

FIG. 3 shows the upper device shell (1) in a top view, wherein the four smaller rubber stoppers (10) above the supporting columns (3) can be seen. On the upper device shell (1), the hinged cover (8) with the hose connection (9) is located, and with the suction hose attached thereto.

FIG. 2 shows the suctioning device (G) in a position rotated by 45°, wherein, in the center of the drawing, the third column (3) of the suctioning device (G) can be seen. FIG. 2 shows the operating position of the folding bellows (4) after the suctioning or pumping of the gaseous medium at the end of the suctioning process.

However, the suctioning process must have occurred first. During the suctioning process, the lower device shell (2) has descended downward due to its own weight on the supporting columns (3), wherein a negative pressure is generated in the internal space (14) of the folding bellows (4). In the process, gas was suctioned from the aid by means of the suction hose. If the weight of the lower device shell (2) by itself is insufficient, then it can be increased with an additional weight (15).

In FIG. 2, the internal space (14) and the individual folding segments (7) have become visible, because the folding bellows (4) has been pulled apart. The number of folding segments (7) will depend on the needs. The folding segments (7) extend between an upper sliding collar (16) visible in FIG. 6, and a lower sliding collar (17). The individual folding segments (7) consist each of two rings (18a and 18b) of a flexible planar material. The material can be plastic or a gas-tight woven fabric. The rings (18a and 18b) are connected to one another on the outer edges (19) of larger diameter and to the flexible planar rings (18a and 18b) of the adjacent folding segments (7) on the inner edges (20) of smaller diameter. In the areas of the material connections, the edges (19 and 20) are provided with reinforcements or stiffenings which are not shown. Such reinforcements or stiffenings can consist of stiff metal hoops, for example.

FIG. 6 shows the folding bellows (4) in a side view. FIG. 7 shows the folding bellows (4) in a top view from the upper side (21) (FIG. 6). One can see the upper sliding collar (16) and the laterally protruding folding segments (7).

FIG. 4 shows a top view onto the lower device shell (2). In the drawing, in this device shell (2) as well, a hinged cover (8a) for closing an emptying opening (8b) for emptying the pump device (G) is provided. This hinged cover (8a), which does not comprise a hose connection (9), can also be omitted.

FIG. 5 shows the suctioning device (G) rotated by 45° with respect to FIG. 1 and turned upside down to be emptied. The lower device shell (2) is now located at the top. The lower device shell (2) has descended due to the force of gravity from the fully filled position of the folding bellows (4) according to FIG. 2 by approximately half of the displacement distance.

The descent was possible since a partial amount of the gaseous medium which has flown in with opened hinged cover (8a) has escaped through an emptying opening (8b). If the hinged cover (8a) and the emptying opening (8b) are not provided, then the medium can be discharged through the hose connection (9), the suction hose (9) or the opening (27) through the device shell (1).

FIG. 8 shows, enlarged, a section A-B according to FIG. 3 through the upper device shell (1) with the supporting columns (3) and the attached folding bellows (4). On both outer sides (22) of the cross-sectional representation, the smaller rubber stoppers (10) are located, which can be used as feet, when the suctioning device (G) is placed upside down for emptying (FIG. 5). The supporting columns (3) are held in guides (23). On the upper side (24) of the device shell (1), in the center area (25), an inlet and outlet opening (27) is provided, which is covered by a securing cross (28) (FIG. 9). The opening (27) is open when the hinged cover (8) is flipped up, and closed when the hinged cover (8) is closed. By means of the securing cross (28), a planar filter (30) is secured in a filter sleeve (29), for example, a cotton pad which has been treated with a disinfecting, sterilizing and odor-absorbing liquid. Under the filter sleeve (29), as plate valve, a flexible valve plate (31) is provided, which can be made of silicone, for example; it arches downward when medium flows in and in the process it lifts off from a valve seat (32). A latching disk (32a) secures the valve plate (31).

The hinged cover (8) is provided with a sealing ring (33) which closes the opening (27) in a gas-tight manner when the hinged cover (8) is closed; after the closing, it rests on the upper side against the seal support (34) (FIG. 9) of the device shell (1). Moreover, the hose connection (9) is located on the hinged cover (8). The suction hose has been removed beforehand. As can be seen in FIGS. 1 and 3, the hose connection (9) consists of a connection which extends at a slant into the hinged cover (8) and is brought up to a bore (35) in which a setting screw (36) limits the amount of air introduced to varying degrees and thus also regulates the descent speed.

The upper portion of the folding bellows (4) is inserted into the upper device shell (1). The device shell (1) has a collar (37) for fastening, onto which the upper sliding collar (16) is slid from below.

A tensioning band (38) clamps the sliding collar (16) firmly on the collar (37). The same fastening is also provided for the lower sliding collar (17) on the lower device shell (2), wherein this lower device shell is slid from above onto the corresponding collar. Under the sliding collar (16), several folding segments are indicated.

FIG. 9 shows the device shell (1) from the upper side (24). In the center area (25), one can see the filter sleeve (29) with the securing cross (28) and the filter (30).

In FIG. 10, a preferred variant of the suctioning device (G) is represented. The folding bellows (4) is partially extended and the hinged cover (8) is open. On the lower device shell (2), bilateral handles (39) are provided, by means of which the lower device shell (2), which has been moved downward, can be pulled up for emptying. In this design, the suctioning device (G) does not have to be turned upside down for emptying. FIG. 11 shows the upper side (24a) of the device variant according to FIG. 10 with the two pull-up handles (39) and with the hinged cover (8) flipped open.

FIG. 12 shows a film bag (40), into which the medium, which has been suctioned into the suctioning device (G) beforehand, is transferred. For this purpose, the film bag (40) has a plug-on crown (41), which, when plugged-on, abut with push-open mandrels (26) through the securing cross (28) of the filter sleeve (29) against the valve plate (31), and, in process, push said valve plate (31) away for opening the valve seat (32). If the film bag (40) is filled, then a closing cover (42) is put onto the plug-on crown (41). The film bag (40) which has been closed in this manner can then be disposed of.

The device has a weight of approximately 4 kg, for example. The suction performance thereof is set, for example, to 2 to 5 mL/minute, for example, 2.5 mL/minute, by a suctioning volume speed limitation, for example, in the form of a regulated or unregulated throttle valve, which can also be designed in the form of small plates with fixed capillary passage. The setting of the regulated valve can be varied via a setting screw (36).

After 2.5 to 3 hours, the medium suctioned therein becomes odorless and, for disposal, it can be transferred by pumping into the film bag (40) with only one hand movement. Subsequently, the suctioning device (G) is again in the starting position and can be connected again.

The suctioning device (G) can be used for numerous purposes in the medical sector and in laboratories, instead of the much more expensive, motor-driven suctioning devices with electronically regulated pumps.

The suctioning device (G) is particularly suitable for the venting device described in the German patent application 102013104659.2 for or as a component of an absorption article for the warm and humid climate existing in the area of an absorption article applied to a patient, such as diapers or a sanitary napkin, for example, and a working method for improving the climate in the absorption article by suctioning of air (preferably without any liquid), from the absorption article with the venting device. To that extent, the subject matter of the German patent application 102013104659.2 is made part of this application by reference also to its disclosure content.

### LIST OF REFERENCE NUMERALS IN THE FIGURES

```
  (1) Upper device shell
  (2) Lower device shell
  (3) Supporting columns
  (4) Folding bellows
  (5) Bellows upper end
  (6) Bellows lower end
  (7) Folding segments
  (8) Hinged cover on upper device shell 1
  (8a) Hinged cover on lower device shell 2
  (8b) Emptying opening in device shell 2
  (9) Hose connection
(10) Small rubber stopper
(11) Large rubber stopper
(12) Handle upper device shell
(13) Handle lower device shell
(14) Internal space of the folding bellows
(15) Additional weight on the lower device shell 2
(16) Upper sliding collar
(17) Lower sliding collar
(18a) Folding segment material rings
(18b) Folding segment material rings
(19) Outer edges of the folding segments
(20) Inner edges of the folding segments
(21) Upper side of the folding bellows
(22) Sides of the upper device shell
(23) Guides for the supporting columns
(24) Upper side of the device shell 1
(24a) Lower side of the variant of the device shell 2
(25) Central area of the device shell 1
(26) Push-out mandrel of the plug-on crown (41)
(27) Opening in the device shell 1 for introduction and discharge
    of medium
(28) Securing cross above the filter
(29) Filter sleeve
(30) Filter made of cotton nonwoven
(31) Valve plate
(32) Valve seat
(32a) Latching disk
(33) Sealing ring
(34) Seal support for the sealing ring 33
(35) Bore
(36) Setting screw
(37) Collar of the device shell 1
(38) Tensioning band
(39) Bilateral pull-up handles
(40) Film bag
(41) Slip-on crown
(42) Closing cover
(G) Suctioning devices
```

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of the device herein described, other than as described in the claims below. It is, therefore, evident that the particular illustrative embodiments disclosed above may be altered or modified and all such variations are considered within the scope and spirit of the present invention. While the embodiments of the device and installation are described in terms of “comprising,” “containing,” or “including” various components, the embodiments also can “consist essentially of” or “consist of” the various components. In particular, every range of values (of the form, “from about a to about b,” or, equivalently, “from approximately a to b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an”, as used in the claims, are defined herein to mean one or more than one of the element that it introduces. If there is any conflict in the usages of a word or term in this specification and one or more patent(s) or other documents that may be incorporated herein by reference, the definitions that are consistent with this specification should be adopted.

1. A device for suctioning gaseous or liquid media, comprising a vertically extendable bellows having a bellows upper end and a bellows lower end, wherein the bellows upper end of the bellows is stationarily fastened to at least one stand, and the bellows lower end of which is connected to a slidably guided loading part which, as the loading part descends due to the force of gravity, spreads out the bellows and in the process generates a negative pressure in the internal space of the bellows, which causes a suctioning of the media from outside via an inlet opening into the bellows, and a valve is provided in the inlet opening, which sets or regulates the suctioning volume speed of the media suctioned through the inlet opening and the descent speed of the loading part.

2. The device according to claim 1, characterized in that the stand comprises three or four vertical columns and supports a rigid lower device shell as loading part, to which the bellows lower end is fastened, wherein the lower device shell, loaded by weight due to the force of gravity, is slidably guided on at least one column, preferably on all the columns, in order to spread out the bellows due to the movement caused by the force of gravity.

3. The device according to claim 1, characterized in that the bellows upper end is fastened to a rigid upper device shell, wherein the upper device shell is stationarily supported by at least one of the columns, preferably by all the columns.

4. The device according to claim 3, wherein the bellows upper end is put from below on a collar of the upper device shell by means of an upper sliding collar, and, independently thereof, the bellows lower end is put from above on a collar of the lower device shell/loading part by means of a lower sliding collar.

5. The device according to claim 1, wherein the bellows is a folding bellows.

6. The device according to claim 5, wherein the folding bellows, between the upper and lower sliding collar thereof, comprises a larger number of folding segments each consisting of two rings of a flexible planar material, wherein the rings are connected on the outer edges of larger diameter to one another and on the inner edges of smaller diameter to the flexible planar rings of the adjacent folding segments, wherein, preferably, at least the areas of the material connections on the outer edges are provided with a stiffening.

7. The device according to claim 2, wherein the bellows comprises the upper device shell or the lower device shell, in particular the upper device shell, the inlet and/or outlet opening, wherein the inlet and/or outlet opening can preferably be closed by means of a hinged cover to be opened upward.

8. The device according to claim 7, wherein in or before the inlet opening, an absorber, filter and/or drying agent are connected, each of which is preferably replaceable.

9. The device according to claim 1, wherein the valve is a regulated or unregulated throttle valve, designed as a body with fixed capillary passage or as small tube with constriction.

10. The device according to claim 3, characterized in that absorber, filter and/or drying agent can be incorporated in the suction hose.

11. The device according to claim 7, characterized in that a receiving bag for receiving suction medium can be connected to the outlet opening.

12. The device according to claim 7, characterized in that the hinged cover is provided with a sealing ring by means of which the inlet and outlet opening is closed when the hinged cover is closed.

13. The device according to claim 1, characterized in that, on the loading part, handles are provided, by means of which the loading part can be pulled up for emptying.

14. The device according to claim 1, characterized in that the medium is a gas, loaded with bodily secretions including urine or faeces, or a fluid which contains or consists of bodily secretions including saliva, wound fluid, urine and/or faeces.

15. An installation comprising:
the device according to claim 1,
a suction hose connected to the device, and
an absorbent body pad,
wherein the suction hose is brought up to the body pad in order to suction medium in the form of a gas from the absorbent body pad by means of the device.

16. The device according to claim 15, wherein the absorbent body pad comprises a diaper, a sanitary napkin or a bandage, and the diaper, sanitary napkin or bandage contain a superabsorber for absorbing moisture and preferably air guide paths which are connected to the suction hose.

17. A method for suctioning gaseous or liquid media with the device according to claim 1 into the bellows, wherein the bellows is spread out due to the movement of the loading part caused by the force of gravity, and the descent speed of the loading part and thus the suctioning volume speed of the media to be suctioned are controlled via a valve.
